(19) United States
(12) Patent Application Publication
(10) Pub. No.: US 2013/0272594 A1
(43) Pub. Date: Oct. 17, 2013

(54) METHOD AND SYSTEM OF IMAGING AND/OR DIAGNOSING A BONE FROM A RECONSTRUCTED VOLUME IMAGE

(71) Applicants: Elazar Zelzer, Rehovot (IL); Tomer Stern, Rehovot (IL); Amnon Sharir, Rehovot (IL); Ron Shahar, Sde Warburg (IL)

(72) Inventors: Elazar Zelzer, Rehovot (IL); Tomer Stern, Rehovot (IL); Amnon Sharir, Rehovot (IL); Ron Shahar, Sde Warburg (IL)

(21) Appl. No.: 13/995,989

(22) PCT Filed: Dec. 21, 2011
(86) PCT No.: PCT/IL11/00957

§ 371 Date: Jun. 20, 2013

Related U.S. Application Data
(60) Provisional application No. 61/425,278, filed on Dec. 21, 2010.

(51) Int. Cl. A61B 6/00

(52) U.S. Cl. CPC A61B 6/5211
USPC 382/131

(57) ABSTRACT

A method of imaging and/or diagnosing intrabony space. The method comprises providing a reconstructed volume image depicting a cylindrical bone, identifying a segment depicting the cylindrical bone in the reconstructed volume image, transforming the segment to generate a three-dimensional (3D) model which aligns a representation of each of a plurality of radial portions having a plurality of different angular orientations of the cylindrical bone in parallel to a common axis, generating at least one coded image mapping of at least one osteo related characteristic of the cylindrical bone along each the radial axis, and outputting the at least one coded image.

### FIELD AND BACKGROUND OF THE INVENTION

The present invention, in some embodiments thereof, relates to method and system of imaging and/or diagnosing a bone and, more particularly, but not exclusively, to a method and system of imaging and/or diagnosing a bone from a reconstructed volume image.

Bone disorders, such as Osteoporosis, do not have a visible expression and therefore hard to detect and to evaluate accurately. Various methods have been developed in the last years for imaging bone characteristics, such as density.

Bone mineral density is measured mainly through the use of x-ray based technologies such as dual energy x-ray absorptiometry (DXA or DEXA) and, to a lesser extent, quantitative computed tomography (QCT). DXA and QCT techniques have many desirable characteristics, such as high sensitivity to the morphology and mineralization of the bone, based on which the risk of fractures can be estimated. Ultrasound also has been shown to be reliable in estimating bone mineral density and predicting the risk of fracture, and has the desirable characteristics of not involving ionizing radiation and using lower cost equipment. In addition, there is a class of x-ray based medical devices called small C-arm fluoroscopes, or simply small C-arms, that typically are used for both surgery and diagnosis of extremities, but can be provided with the additional capability of making bone assessment measurements.

As an example of commercially available products of this type, Hologic, Inc. of Bedford, Mass. offers a line of DXA equipment under the trade names ACCLAIM and QDR and model designations such as 4500 and 1000, an ultrasound bone sonometer under the trade name Sahara, and a small C-arm under the trade name Fluoroscan followed by model designations.

During the last year various methods and systems have been developed. For example, US Patent Application No 2002/0022779 describes a compact magnet and an RF probe which can accommodate a human extremity such as a heel are used to construct a compact MRI system for diagnosis and follow-up of osteoporosis and other diseases. Methods for measuring and calculating proton density in inhomogeneous static magnetic field, magnetic field gradients, and RF magnetic field are provided using 2D spin-echo image acquisitions with external reference materials and image analyses. The measured proton density of bone marrow is used for computation of trabecular bone volume fraction, which can be used for diagnosis of osteoporosis and other diseases.

Another example is described in US Patent Application No. 2010/0142675 describes a method and system for determining the bone mineral density of a body extremity. An image of a body extremity is acquired using a mammography x-ray system whereby a bone mineral density inspection can be performed on the image. The system for determining the bone mineral density of a body extremity includes: a support for supporting the body extremity; a detector for capturing an image of the body extremity; and an x-ray source adapted to project an x-ray beam through the body extremity toward the detector, the x-ray source having a voltage of no more than about 45 kVp and having a target/filter combination of rhodium/rhodium, molybdenum/molybdenum.

### SUMMARY OF THE INVENTION

According to some embodiments of the present invention there is provided, a method of imaging and/or diagnosing intrabony space. The method comprises providing a reconstructed volume image depicting a cylindrical bone, identifying a segment depicting the cylindrical bone in the reconstructed volume image, transforming the segment to generate a three-dimensional (3D) model which aligns a representation of each of a plurality of radial portions having a plurality of different angular orientations of the cylindrical bone in parallel to a common axis, generating at least one coded image mapping of at least one osteo related characteristic of the cylindrical bone along each the radial axis, and outputting the at least one coded image and/or a numeral value quantifying one or more properties of the cylindrical bone.

Optionally, the at least one coded image is color coded.

Optionally, the outputting comprising presenting the at least one coded image.

Optionally, the method further comprises analyzing the at least one coded image to automatically diagnose at least one osteo related pathology.

Optionally, the method further comprises registering at least the segment onto a nonlinear cylindrical space, the transforming comprising mapping the plurality of radial portions by a transformation from the continuous nonlinear cylindrical space to a continuous Euclidean space.

Optionally, the generating comprising calculating a bone mineral content (BMC) along each the radial portion.

Optionally, the generating comprising calculating an unweighted bone mineral content (uwBMC) along each the radial portion.

Optionally, the generating comprising calculating a bone mineral density (BMD) along each the radial portion.

Optionally, the generating comprising calculating an unweighted bone mineral density (uwBMD) along each the radial portion.

Optionally, the generating comprising calculating the distance from the center of the Bone marrow to the Endosteum (Endosteal thickness) along each the radial portion.

Optionally, the generating comprising calculating the thickness of the Cortex (Cortical thickness) along each the radial portion.

Optionally, the generating comprising calculating the distance from the center of the Bone marrow to the Periosteum (Periosteal thickness) along each the radial portion.

Optionally, the generating comprising calculating the fraction of volume that is not mineralized along each the radial portion, including only the part between the endosteum and the periosteum, referred to as Cortical Porosity. For example, the volume along the axis which includes marrow (no mineral) divided by the total volume along the axis (both with and without mineral) from the endosteum to the periosteum (i.e. without a portion from the center of the bone to the endosteum).

Optionally, the generating comprising calculating the fraction of axis length that is not mineralized along each the radial portion, including only the part between the endosteum and the periosteum (referred to as unweighted Cortical Porosity).

Optionally, the generating comprising calculating the at least one osteo related characteristic along each the radial axis as a function of a plurality of radiodensity units of respective voxels in at least the segment.

Optionally, the reconstructed volume image is obtained by a computed tomography (CT) scanning.

Optionally, the at least one osteo related characteristic comprises morpho-density characteristics.

Optionally, the method further comprises diagnosing an osteoporotic level of the cylindrical bone by analyzing the at least one coded image.

Optionally, the method further comprises detecting at least one metastatic or primary tumor site in the cylindrical bone by analyzing the map.

Optionally, the plurality of radial axes are perpendicular to a longitudinal axis of the cylindrical bone.

Optionally, the transforming emulates the spreading of the cortex layer of the segment on a plane.

Optionally, the reconstructed volume image depicting a plurality of cylindrical bones the identifying, transforming, generating, and outputting are iteratively repeated with each the cylindrical bone.

According to some embodiments of the present invention there is provided, a system of imaging intrabony space. The system comprises an interface which receives a reconstructed volume image depicting a cylindrical bone, a computing unit which transforms a segment depicting the cylindrical bone in the reconstructed volume image to generate a three-dimensional (3D) model which aligns a representation of each of a plurality of radial axes having a plurality of different angular orientations of the cylindrical bone in parallel to a common axis and generates at least one coded image mapping of at least one osteo related characteristic of the cylindrical bone along each the radial axis, and a display which outputs the at least one coded image.

Optionally, the interface receives the reconstructed volume image from a member of a group consisting of: a computer aided diagnosis (CAD) module, a client terminal of a physician, a central database, a DICOM server, a radiology information system (RIS), a hospital information system (HIS), and a clinical information system (CIS).

According to some embodiments of the present invention there is provided, a method of diagnosing an intrabony space. The method comprises providing at least one coded image mapping a sum of values of at least one osteo related characteristic in each of a plurality of portions of at least one bone of a patient, analyzing a pattern depicted in the at least one coded image to identify a pathological expression, and outputting an outcome of the analysis.

Optionally, the sum is weighted.

More optionally, the method further comprises calculating the osteo related characteristic per voxel in least one reconstructed volume image of the at least one bone, the osteo related characteristic is selected from a group consisting of a bone mineral content (BMC), an unweighted bone mineral content (uwBMC) a bone mineral density (BMD), an unweighted bone mineral density (uwBMD), a value for calculating a Cortical Thickness, a value for calculating Endosteal Thickness, a value for calculating Periosteal Thickness, a value for calculating Cortical Porosity, a value for calculating unweighted Cortical Porosity.

Optionally, the analyzing comprises: selecting at least one 2D reference map according to at least one demographic characteristic of the patient, and matching between the at least one 2D reference map and the at least one coded image.

Optionally, the at least one coded image comprises a plurality of coded images, the analyzing comprises matching between the plurality of coded images to identify a difference and performing the analysis according to the difference.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a method of imaging and/or analyzing intrabony space of cylindrical bones, according to some embodiments of the present invention;

FIGS. 2A-2B are schematic illustrations of an exemplary cylindrical bone in pre transformed and transformed states, according to some embodiments of the present invention;

FIGS. 3A-3B depict a cross section of a 3D segment of a bone depicted in a CT image and the cross section after having been transformed, generated according to some embodiments of the present invention;

FIG. 4 is a flowchart of a process for constructing a nonlinear cylindrical grid, according to some embodiments of the present invention;

FIG. 5A is a schematic illustration of exemplary unrolled image maps which are generated using different operators, from the bones shown in FIG. 5B, according to some embodiments of the present invention;

FIG. 5B is a schematic illustration of Ulna bones of a wild type (WT) mouse and a bone morphogenetic protein 2 (BMP2) conditional knockout (K.O.) mouse;

FIG. 5C is a schematic illustration of other exemplary coded maps (images) of a Tibia bone having a BMP4 manipulated gene, which are generated using different operators, according to some embodiments of the present invention;

FIG. 6 is a set of exemplary coded images, unrolled image maps, generated from two human Humeri bones of a healthy patient (left) and an Osteoporotic patient (right) which are indicative of BMD in the entire proximal ends (˜3.5 cm) of the bones, according to some embodiments of the present invention;

FIGS. 7A, 7B, and 7C depict additional exemplary coded images, unrolled image maps, of the same Humeri bones as in FIG. 6, which are generated using different operators, according to some embodiments of the present invention;

FIGS. 8A-8C and FIGS. 9A-9C are coded images, unrolled image maps, of left and right femur bones of a healthy patient and a cancerous patient, generated according to some embodiments of the present invention;

FIG. 10 is a flowchart of a method of diagnosing an intrabony space, according to some embodiments of the present invention; and

FIG. 11 is a schematic illustration of an imaging system of imaging intrabony space of a cylindrical bone, for example by implementing the method depicted in FIG. 1, according to some embodiments of the present invention.

### DESCRIPTION OF EMBODIMENTS OF THE INVENTION

The present invention, in some embodiments thereof, relates to method and system of imaging and/or diagnosing a bone and, more particularly, but not exclusively, to a method and system of imaging and/or diagnosing a bone from a reconstructed volume image.

According to some embodiments of the present invention, there are provided methods and systems of diagnosing intrabony space by analyzing a pattern depicted in a coded image of one or more intrabody osteo related characteristics of portions of a bone of patient. The osteo related characteristic may be a bone mineral content (BMC), an unweighted bone mineral content (uwBMC), a bone mineral density (BMD), an unweighted bone mineral density (uwBMD), the Cortical Thickness of the bone, and/or any other characteristics of the bone which may be extracted from the transformed 3D image. The coded image is optionally extracted from a segment depicting a bone of a patient in a reconstructed volume image, such as a CT image. The analysis is optionally performed by comparative analysis, which may be referred to herein as matching, between the pattern depicted in the coded image and a pattern depicted in a 2D reference map. The 2D reference map is optionally selected from a database which includes a plurality of reference models, each depicts an exemplary pattern associated with a different demographic group of patients. The demographic groups may be divided according to different age, gender, race, pathology, and/or medical condition. The analysis of the coded image of the patient allows identifying local indicators in the different volumes of the depicted pattern.

According to some embodiments of the present invention, there are provided methods and systems of imaging and/or diagnosing intrabony space by transforming a segment depicting a cylindrical bone in a reconstructed volume image, such as a CT image, to generate a three-dimensional (3D) model that aligns a representation of each of a plurality of radial portions, having a plurality of different angular orientations, of the imaged cylindrical bone in parallel to a common axis, for example from a regular Euclidean space, by defining a Nonlinear-Cylindrical coordinate system on the Euclidean space (i.e. on the pre-transformed image), and representing the Nonlinear-Cylindrical coordinate system using a quasi-Euclidean space. The term quasi is used as although data points are organized in a regular 3D grid, measures like distances, angles between coordinates and the volume of voxels composing this space is not calculated as in a regular Euclidean space. As used herein a radial portion means a linear segment, which is one of some number of linear segments radiating from a longitudinal axis, linear, curved, or graded, of a bone, connecting the longitudinal axis with the bone surface or with some other point outside of the surface of the bone.

According to some embodiments of the present invention, there are provided a method of imaging and/or diagnosing intrabony space that is based on such a reconstructed volume image that depicts a cylindrical bone. The received reconstructed volume image is processes to identify a segment that depicts the cylindrical bone in the reconstructed volume image. The segmented bone is then transformed to a target space, such as Euclidean space where a plurality of radial axes of the imaged cylindrical bone are aligned in parallel to a common axis. This allows generating one or more maps which map the osteo related characteristics of the cylindrical bone along each radial axis and outputting these map(s) to a display and/or a processing unit. Such maps allow manual and/or automatic diagnosis of a bone segment in a reconstructed volume image, which may be understood as an unsegmented image of a bone and a mask defining location, size, shape, and/or orientation the bone segment, and/or a portion thereof, for example for estimating the presence and/or absence of metastatic sites and/or osteoporotic level.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

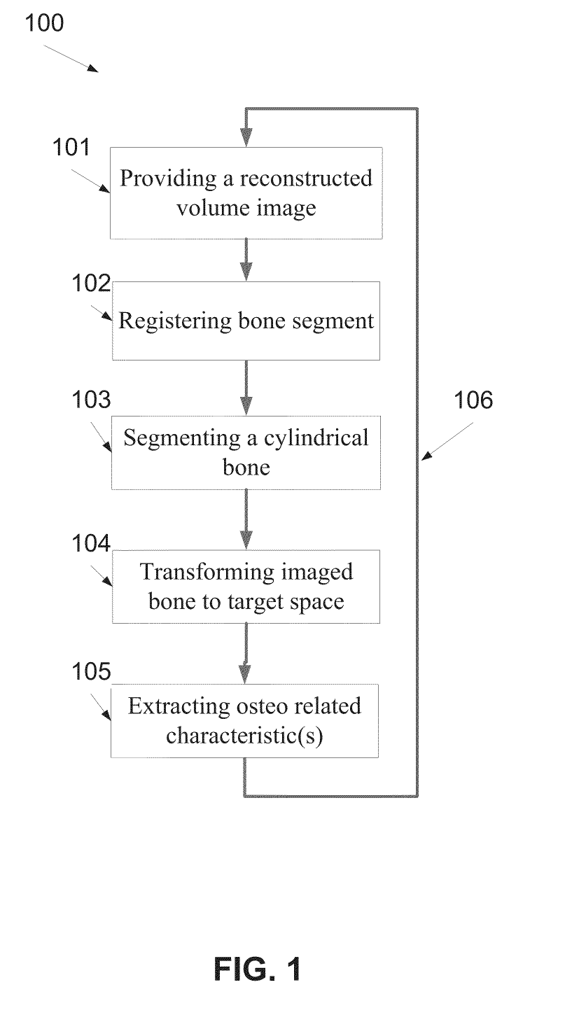

Reference is now made to FIG. 1, which is a flowchart 100 of a method 100 of imaging and/or analyzing intrabony space of cylindrical bones, according to some embodiments of the present invention. The method 100 includes a transformation that is based on the morphology of the cylindrical bones, such as the Femur and the Tibia and/or cylindrical portions of bones, such as the Radius, the Ulna, the Humerus, the Femur, the Tibia, the Fibula, the ribs, and the like.

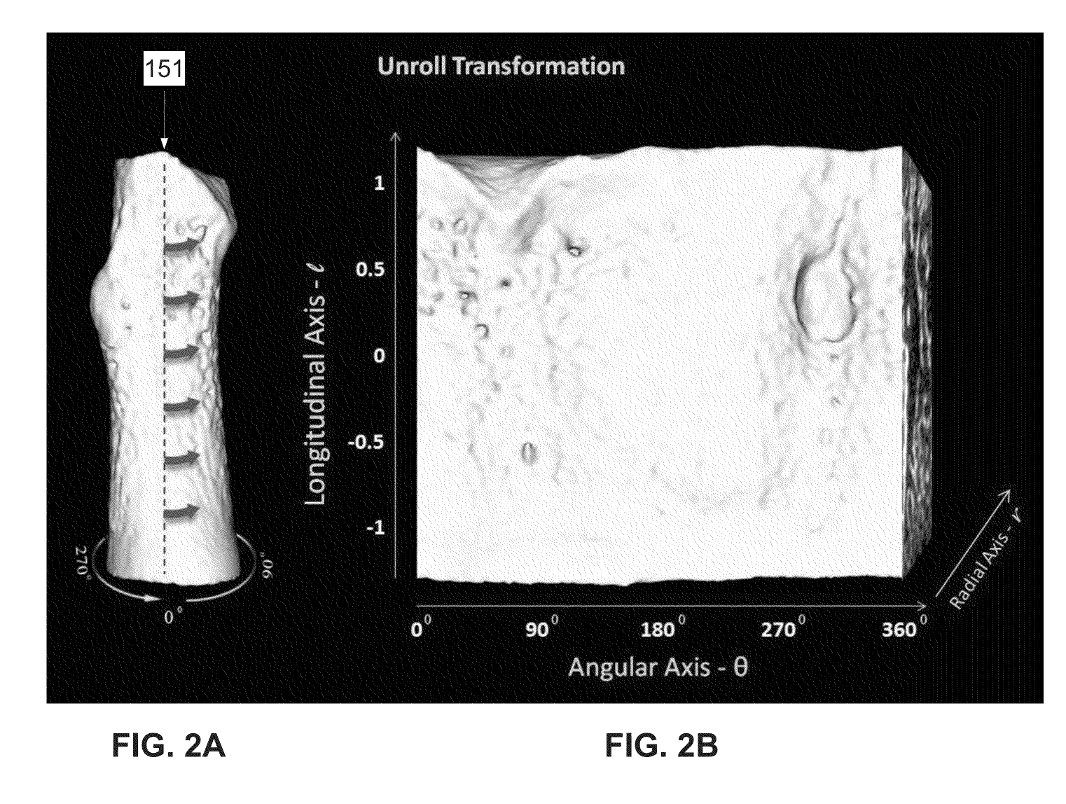

Reference is now also made to FIGS. 2A-2B, which are schematic illustrations of an exemplary cylindrical bone in pre transformed and transformed states, according to some embodiments of the present invention. These figures figuratively illustrate a transformation by the method which may be performed by identifying and/or estimating a longitudinal central axis of a bone, optionally using a model or a volume center estimation in different cross sections, followed by performing a virtual cut on the cortex of the cylindrical bone along its entire length from a predefined angle, for example as shown at 151, referred to herein as a perspective angle, and unrolling the entire cortex of the bone from the cut, about the longitudinal central axis to obtain a coded image which is based on an image of an unrolled bone, for example as depicted in FIG. 2B. As used herein, a coded image, which may be referred to as a coded map, is an image which is optionally coded according to the weighted sum of values of one or more osteo related characteristics of each of a plurality of portions of the imaged bone. The coding is optionally in colors and/or hues, where each color or hue is selected according to the weighted sum of values of one or more osteo related characteristics of each of a plurality of portions of the imaged bone. A coded image may also be coded in gray scale colors. Each color or hue represents a different value. Each value is based on a weighted sum of one or more osteo related characteristics of voxels of a portion of the imaged bone. The coded image may be a topographic map wherein the depicted height represents a value. The coded image may be a processed map wherein areas in which the weighted sum of values of one or more osteo related characteristics of is above a certain threshold level.

A figurative image of an exemplary bone and an unrolled version of this exemplary bone are provided in FIGS. 2A and 2B. For clarity, this is a non limiting schematic example as the intrabony space may be imaged in various ways, as described below.

First, as shown at 101, a reconstructed volume image having a three dimensional (3D) segment representing at least a portion of an intrabony space of one or more cylindrical bones is provided. As used herein, a reconstructed volume image means an image obtained by computed tomography (CT) scanning, an image obtained by magnetic resonance imaging (MRI) scanning, an image obtained by positron emission tomography (PET)-CT scanning, and/or any other medical scanning modality. The 3D segment is optionally a set of voxels represented in the reconstructed volume image.

Optionally, as shown at 102, the 3D bone which is imaged in the provided reconstructed volume image is registered to a known spatial position in an Euclidean space. Optionally, the registration is performed by a pairwise/multiple image registration method. First, a crude segmentation using a global threshold is applied in order to enable extraction of morphological bone features and better the distinction between bone and non-bone voxels. Then circumferential features of the bone are extracted, based on which the initial approximation of the registration is performed. During the initial approximation stage, several transformations are considered, and the one having the optimal score is taken for fine tuning. Finally, a fine tuning step takes place based on a volume based registration module until convergence is reached to a desired level. An optional way to generalize the method for multiple bones is by performing multiple pairwise alignments between all pairs of sufficiently similar bones, constructing a Minimum Spanning Tree from the obtained results based on the scores of the pairwise registrations, and transforming each bone image based on its location on the spanning tree relatively to a predefined “root” image.

Optionally, as shown at 103, a cylindrical bone which is represented in the reconstructed volume image is segmented, referred to herein as a segmented bone. The segmentation may be performed using known bone segmentation algorithms, for example see Y. Kang, K. Engelke, and W. A. Kalender, “A new accurate and precise 3-D segmentation method for skeletal structures in volumetric CT data,” IEEE Trans. Med. Imag., vol. 22, no. 5, pp. 586-598, May 2003, V. Grau, U. U. J. Mewes, M. Alcaniz, R. Kikinis, and S. K. Warfield, “Improved watershed transform for medical image segmentation using prior information,” IEEE Trans. Med. Imag., vol. 23, no. 4, pp. 447-458, April 2004, A. Elmoutaouakkil, E. Peyrin, J. Elkafi, and A. Laval-Jeantet, “Segmentation of cancellous bone from high-resolution computed tomography images: Influence on trabecular bone measurements,” IEEE Trans. Med. Imag., vol. 21, no. 4, pp. 354-362, April 2002, T. B. Sebastian, H. Tek, J. J. Crisco, and B. B. Kimia, “Segmentation of carpal bones from CT images using skeletally coupled deformable models,” Med. Image Anal., vol. 7, pp. 21-45, 200, X. M. Pardo, M. J. Carreira, A. Mosquera, and D. Cabello, “A snake for CT image segmentation integrating region and edge information,” Image Vis. Comput., vol. 19, pp. 461-475, 2001, and K. Haris, S. Efstratiadis, and A. Katsaggelos, “Hybrid image segmentation using watersheds and fast region merging,” IEEE Trans. Image Process., vol. 7, no. 12, pp. 1684-1699, December 1998, which are incorporated herein by reference.

Now, as shown at 104, a transformation which maps each coordinate of the bone segment into a coded image, optionally in a quasi-Euclidean target space. For example, the transformation constructs a nonlinear grid based on control points that are described below so that unsegmented and binary masks are transformed. The transformation optionally defines a Nonlinear-Cylindrical coordinate system on the regular Euclidean space of the original CT image, and then represents the image in its Nonlinear-Cylindrical coordinate system based on a quasi-Euclidean space. This transformation allows, as shown at 104, generating one or more coded images which are indicative of a value of one or more osteo related characteristics of each of a plurality of radial portions of the imaged bone. As used herein, a radial portion means a portion of the bone along a straight line between the main longitudinal axis of the bone and the outer surface of the cortex and/or along a line which bisects or otherwise divides the body of the imaged bone. As used herein an osteo related characteristic means a bone mineral content (BMC) along the radial portions of the bone, a bone mineral density (BMD) along the radial radial portions of the bone, the Cortical Thickness of the bone (as the distance from the Endosteum to the Periosteum of the bone), and/or any other characteristics of the depicted bone which may be extracted from the imaged bone segment. Optional measuring units for density related characteristics are the Hounsfield Units (HU) and g/cm̂3.

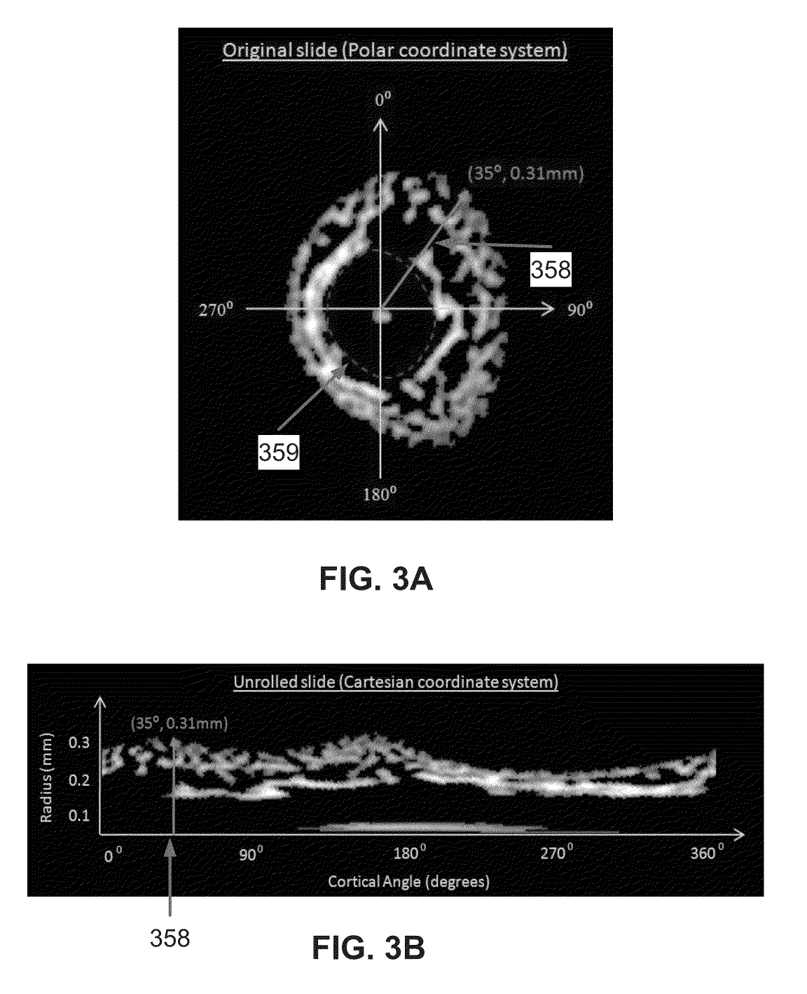

FIGS. 3A and 3B depict a 2D simplification (without considering the longitudinal axis) of the method with a single cross section of an embryonic day 18 Femur bone of a WT mouse (top) and the corresponding transformed section (bottom). Ignoring the influence of the longitudinal axis, optionally, the transformation is carried out by setting a 2D polar coordinate system, defined by angular and radial axes with an origin superimposed on the geometric center of the putative region formerly occupying the cartilaginous model of the bone and marked by a dashed line 359. The transformed slide, depicted in FIG. 3B, may be obtained by representing the transversal slide, depicted in FIG. 3A in a quasi-Euclidean space defined by X and Y axes with the pre-transformation angular axis replaced by a post-transformation X axis and the pre-transformation radial axis is replaced by a post-transformation Y axis. For example, the arrow 358 depicted in FIG. 3A is a radial portion having a length of 0.31 mm and an orientation of 35° from axis 0°. After having been transformed, the entire line of voxels lying about it (based on the nonlinear cylindrical coordinate system) is located perpendicularly to the 35° of the X axis, with length of 0.31 mm. The representation depicted in FIG. 3B allows performing simple angle-wise calculations in a quasi-Euclidean space and imaging the cross section in an informative manner, emphasizing important morphological aspects, like the distance between the Periosteum of the bone and the geometric center of the cartilaginous model at each direction. The obtained quasi-Euclidean space can be viewed as a data structure for simplifying the design and execution of complex calculations originally built for a non-linear cylindrical coordinate system by representing it using a simple 3D matrix.

Reference is now made to a mathematical description of an exemplary transformation that allows mapping the coordinates of a 3D segment depicting a cylindrical bone from a reconstructed volume image into a transformation space.

Optionally, the transformation is a [symbol] ³→ [symbol] ³ transformation that is optionally a deforming, yet reversible, information preserving, transformation, defined on 3D images of long bones and their binary masks as obtained from optionally a segmentation procedure.

Optionally, the transformation is performed from the original Euclidean space on which a nonlinear cylindrical coordinate system is defined, to a quasi-Euclidean space representing the nonlinear cylindrical system. For brevity, l denotes a longitudinal/curvature axis, θ denotes angular axis/azimuth, and r denotes a radial axis. The Nonlinear-Cylindrical coordinate system is optionally based on grid lines that determine the boundaries of non-cubic voxels that assemble the Nonlinear-Cylindrical space. This also allows calculating the intensity and the volume/weight of each newly formed non-cubic voxel. Once this is done, the transformation may be performed by assigning the resulting intensities and volumes into two regular 3D matrices (the quasi-Euclidean representation of the Nonlinear-Cylindrical system) indexed by (l, 0, r), where one is for the intensities of the non-cubic voxels and the other is for the weight/volume of the non-cubic voxels.

Optionally, the transformation, is defined by the following algorithm:

### INPUT:

receiving Iε [symbol] ^{x,y,z} where I denotes a reconstructed volume image such as an unsegmented image of a long bone segment;

receiving I^{bw}ε{0,1}^{x,y,z} where I^{bw} denotes a binary image obtained by binarizing l into bone (1) and background (0) using one of the aforementioned segmentation process;

receiving control points CP=(cp₁, . . . , cpₙ) where CP denotes a series of n 3D control points, sampled from the putative location of the longitudinal centre of the cartilaginous model prior its ossification (this is optionally done manually by an expert anatomists). Optionally, the order of the points along the length of the bone is from distal-most (cp₁) to proximal-most (cpₙ); and
receiving ur:yinε{1, . . . , n} where origin denotes an index of the control point that marks the putative location of the anatomic origin of the entire diagnosed bone or diagnosed bone portion, for example the anatomic point around which the bone collar was formed;

### OUTPUT:

outputting Uε [symbol] ^{l,θ,r} where U denotes a grayscale image of I after having been transformed and segmented, which may be referred to herein as unrolled; and

outputting ωε [symbol] ^{l,θ,r} where ω_{xyz} denotes a weight matrix, with the volume calculated for the non-cubic voxel U_{xyz}, for example at the constructed quasi-Euclidean space.

### TRANSFORMATION:

Optionally, a transformation, as shown at 302, is performed by constructing a spline, for example using a Polynomial Piecewise Function, from the series CP=(cp₁, . . . , cpₙ), where the knot vector is defined to be: t=(1, . . . , n). In such embodiments, following the construction of the spline, C is re-parameterized with lε [symbol] that is defined as:

$$C ( l ) = \{ \begin{matrix}C ( t ) \vert Arc Length ( C ( origin ) -> C ( t ) ) = l ⋀ t > origin & l > 0 \\ C ( origin ) & l = 0 \\ C ( t ) \vert Arc Length ( C ( origin ) -> C ( t ) ) = - l ⋀ t < origin & l < 0\end{matrix}$$

where C(l) denotes the point on the spline that is l units from a point of an anatomic origin toward the proximal (distal) part of the bone if l is positive (negative), or for example the anatomic origin itself if l=0, as measured by the ArcLength of C. This parameterization provides a natural and an intuitive association between the anatomy of the bone and its mathematical representation. To implement the re-parameterization, the correspondence between l and t is inferred based on a function from l to t by taking a dense and uniformly spaced sample of values over the entire range of t, where 0<dt<<1. This allows estimating the following:

L(tᵢ)=ArcLength(C(1)→C(tᵢ))−ArcLength(C(1)→C(origin))

for each tᵢ in the sample by approximating ∫₁^{t}^{ᵢ}∥C′(τ)∥dτ, for example with a recursive adaptive Simpson quadrature. Now, the coordinates of the curvature at any given value l is calculated, for example using obtained pairs (L(tᵢ), tᵢ) in order to interpolate for the corresponding value t of the original parameter—t, and provide it as an argument to C(t).

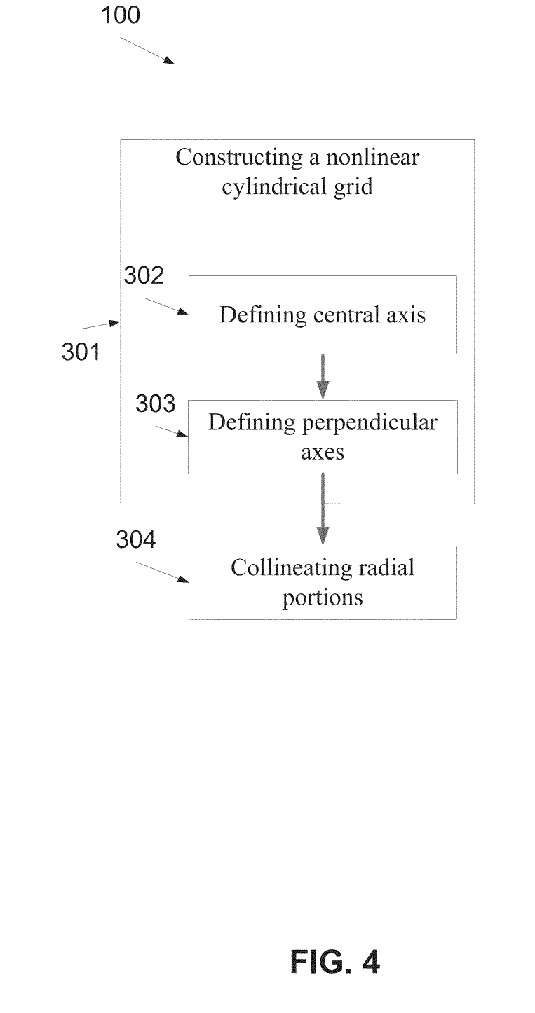

The transformation allows constructing a nonlinear cylindrical grid. Reference is now made to FIG. 4, which is a flowchart of a process for constructing a nonlinear cylindrical grid, according to some embodiments of the present invention. First, as shown at 301, optionally a single 2D polar coordinate system is established on a plane, which is normal to C(l) for each value of l, referred to herein as a normal plane from each point on C(l), where g lε [symbol] . The single 2D polar coordinate system at a given point of C(l) may be established by setting the origin at C(l) and constructing two orthogonal unit vectors, denoted herein as [symbol] (l) and [symbol] (l) that serve as the 0° and 90° axes, on the normal plane, respectively.

Now, as shown at 303 the pointing direction of the 0° axis, which may be referred to as the primary axis, on the normal plane is now defined. Figuratively, this definition is of where the cortex of the imaged bone and/or portion of the imaged bone is virtually cut to initiate the unrolling. Then, after the orientation of the 0° axis is set, the pointing direction of the 90° axis, which may be referred to as the perpendicular axis is set. This setting defines the direction to which the bone is unrolled from the 0° axis, which may be referred to herein as a virtual cut, for example clockwise and/or counterclockwise, assuming a predefined convention.

Optionally, the setting of these axes is done by a scheme in which the axes are not determined exclusively by the bone curvature (like in the Frenet-Serret formulas) but also according to the spatial position of the bone on the XYZ coordinate system. In this embodiment, all 0° axes are collinear with the XZ plane while pointing toward increasing X values, and the 90° axes point toward increasing Y values, assuming a relatively linear structure of the bone.

In order to allow such collineation, as shown at 304, [symbol] (l) and [symbol] (l) are set to meet the following:

1. [symbol] (l), [symbol] (l) and T(l) are mutually orthogonal;
2. ∥ [symbol] (l)∥=∥ [symbol] (l)∥=1.
3. [symbol] _{Y}(l)=0, [symbol] _{X}(0)≧0, so as to define the direction of the cutting line, which is the axis from which the segmented bone is figuratively unrolled; and
4. [symbol] _{Y}(0)≧0 so as to define the direction of unrolling from the cutting line.

Based on these requirements [symbol] (l) and [symbol] (l) are uniquely defined as: Let Ĉ(l)=<C_{X}(l), 0, C_{Z}(l)> be the orthogonal projection of C(l) on the XZ plane, let \hat{T}(l) be the tangent of Ĉ at Ĉ(l), and let T(l) be the tangent of C at C(l), then (with the exception of: sign(0)=1):

[symbol]

(l)=<\hat{T}_{Z}(l)>·sign(C′_{Z}(0))

[symbol]

(l)=T(l)× [symbol] (l)·sign(C′_{Z}(0))

Now, based on the vectors C(l), [symbol] (l), and [symbol] (l) the transformation between a continuous Euclidean space and a continuous quasi-Euclidean space (via an intermediate nonlinear cylindrical coordinate system) may be defined as follows:

\dot{U}(l,θ,r)=l(x,y,z) [symbol] (x,y,z)=C(l)+r(Cos(θ) [symbol] (l)+Sin(θ) [symbol] (l))

Optionally, voxel oriented calculations are facilitated by defining the domain of the voxel U(l, θ, r) as:

D_{l,θ,r}={\dot{U}(l₁,θ₁,r₁)|l₁ε(l−1,l],θ₁ε(θ−1,θ],r₁ε(r−1,r]}

for lε [symbol] , θε{1, . . . , 360} and rε [symbol] .

Optionally, the volume, or an approximation thereof, may be calculated for some or all of the voxels using a volume (triple) integral over the 3D domain. For example, an approximation may be calculated considering the volume of the hexahedron bounded by the 8 nodes/corners of the domain as a convex-hull (or 6 if r=1, in which case the voxel becomes a triangular prism), see Efficient Computation of Volume of Hexahedral Cells, J. Grandy, Oct. 30, 1997 which is incorporated herein by reference. Optionally, the intensity, or an approximation thereof, may be calculated for some or all of the voxels. The natural point of each voxel to be interpolated is at \dot{U}(l−0.5, θ−0.5, r−0.5), yet to correspond with the described approximation of the volume, the value is optionally interpolated at the geometric center of the obtained convex hull using for example trilinear interpolation or nearest-neighbor interpolation.

It should be noted that a similar transformation may be applied on I^{bw} where 1=bone and 0=background in order to segment the unrolled bone in the obtained image, for example as described above. This is optionally done without calculating the volume of each voxel again, and optionally using nearest-neighbor interpolation to interpolate the binary intensities/labels of 0/1. In such a manner, a corresponding binary mask of the unrolled image is formed and can be used to segment the bone after the transformation is applied, optionally by zeroing I at where I^{bw}=0.

In a figurative manner, the relations between the continuous nonlinear cylindrical grid and the quasi-Euclidean space may be described as a shift between two 3D data representations, namely from a Nonlinear-Cylindrical coordinate system that the algorithm constructs uniquely for the specific bone within the original image—on which calculations may be very complicated, to a quasi-Euclidean space, where the originally curved curvature line of a cross section of the imaged bone is linearized to form the new Y axis, the circular/angular axes of the bone are linearized to form the new X axis, and the radial axes of the bone, each between the central axis and a point at a specific angle along the curvature line, form the new depth axis—Z, on which calculations (as will be demonstrated) can be made immeasurably simpler. This way, the encircling surface of the imaged bone or portion, which may be understood as a 360° envelope, internal-most to external-most faces the point of view of the user, while the voxels which depict the rest of the cortical wall and are followed by the voxels which depict the trabeculae and the marrow, if they are visible in the processed reconstructed volume image, reside deeper along the Z axis, figuratively behind the surface as shown at FIG. 2B (see region above the 360°).

Now, as shown at 105, the coded image, which is optionally based on a transformed image of the unsegmented input image, which may be referred to herein as an unrolled bone image and optionally defined by the quasi-Euclidean space, is analyzed to extract one or more osteo related characteristics, such as morpho-density characteristics, of the imaged bone. Optionally, the one or more osteo related characteristics are extracted per radial portion.

Optionally, one or more mathematical operators (O) are applied on the data in the unrolled bone image, for example in order to study the osteo related characteristics of one or more of the medullary cavity, the cortex, the endosteum, and/or the periosteum, along the radial axes of the imaged bone, for example the thickness and/or the density thereof. The operators enable calculating and imaging information which is optionally associated with the distances between anatomically meaningful landmarks and/or density along the radial portions, while combining the information in some way. to form composite parameters.

Optionally, one or more of the following osteo related characteristics each of the radial portions of the transformed bone, in different angles from the main longitudinal axis, are extracted using designated operators. For brevity, in Uε [symbol] ^{l,θ,r}, ωε [symbol] ^{l,θ,r} denotes a weight matrix where r=(1, . . . , r) and [dv, dv, dv] denotes voxel dimensions in a pre-transformation image I. Each operator returns an unrolled image map, which is a 2D matrixε [symbol] ^{l,θ}.
1. Bone Mineral Content—based on a bone mineral content (BMC) map: [symbol] [l, 0]=dv³ Σᵣ U[l, 0, r]ω[l, 0, r].
2. Unweighted Bone Mineral Content—based on an unweighted bone mineral content (uwBMC) map:
3. uw [symbol] [l, θ]=dv³ Σᵣ U[l, θ, r]. In this operator the densities are not weighted by the volumes of the voxels. This unweighted version provides a somewhat different perspective on the distribution of densities that does not include the anatomy of the analyzed region.
4. Bone Mineral Density—based on a bone mineral density (BMD) map:

$$BMD [ l , θ ] = \frac{{\sum }_{r} U [ l , θ , r ] ω [ l , θ , r ]}{{\sum }_{r} ω [ l , θ , r ]} .$$

5. Unweighted Bone Mineral Density—based on an unweighted bone mineral density (uwBMD) map:

$$uw BMD [ l , θ ] = \frac{{\sum }_{r} U [ l , θ , r ]}{{\sum }_{r} 1} .$$

To characterize the morphology of bone I, optionally Iₚₑᵣε{0,1}^{x,y,z} is defined as Iₚₑᵣ[x, y, z]=1 iff the voxel [x, y, z] resides within the volume enclosed by the bone's Periosteum and the growth-plates, and Uₚₑᵣε{0,1}^{l,θ,r} optionally as Iₚₑᵣ after having been unrolled. Optionally, the following are respectively defined: I_{end}ε{0,1}^{x,y,z} and U_{end}ε{0,1}^{l,θ,r} based on the Endosteum of the bone. In addition, we define the Boolean operator mineralized in the following manner:

$$mineralized [ l , θ , r ] = \{ \begin{matrix}0 & U [ l , θ , r ] = 0 \\ 1 & otherwise .\end{matrix}$$

Note that to utilize these definitions proper segmentation that separates between cortical and trabecular bone may be used.
6. Periosteal thickness—based on a map showing the distances of the Periosteum from the curvature line: Thickₚₑᵣₗ[l,θ]=dv Σᵣ U_{endo}[l, θ, r].
7. Endosteal thickness—based on a map showing the distances of the Endosteum from the curvature line: Thick_{endo}[l,θ]=dv Σᵣ U_{endo}[l, θ, r].
8. Cortical thickness—based on a map showing the thickness of the cortex (from Endosteum to Periosteum): Thick_{cort}[l,θ]=Thickₚₑᵣₗ[l,θ]−Thick_{endo}[l,θ].
9. Cortical porosity—based on a map showing the porosity of the cortex (from Endosteum to Periosteum):

$$Porosity [ l , θ ] = \frac{{\sum }_{r} ω [ l , θ , r ] \times mineralized ( l , θ , r )}{{\sum }_{r} ω [ l , θ , r ]}$$

10. Unweighted cortical porosity—based on a map showing the unweighted porosity of the cortex (from Endosteum to Periosteum):

$$uwPorosity [ l , θ ] = \frac{{\sum }_{r} mineralized ( l , θ , r )}{{\sum }_{r} 1} .$$

One of the advantages of analyzing the data in the unrolled image is that the relatively planar representation of the bone enables to calculate numerous mineralization and morphological characteristics at each sectional segment of the entire bone in a straightforward calculation and/or to visualize the results as 2D topographic maps. This allows detecting minute phenotypic alterations caused by a genetic origin and/or an environmental origin.

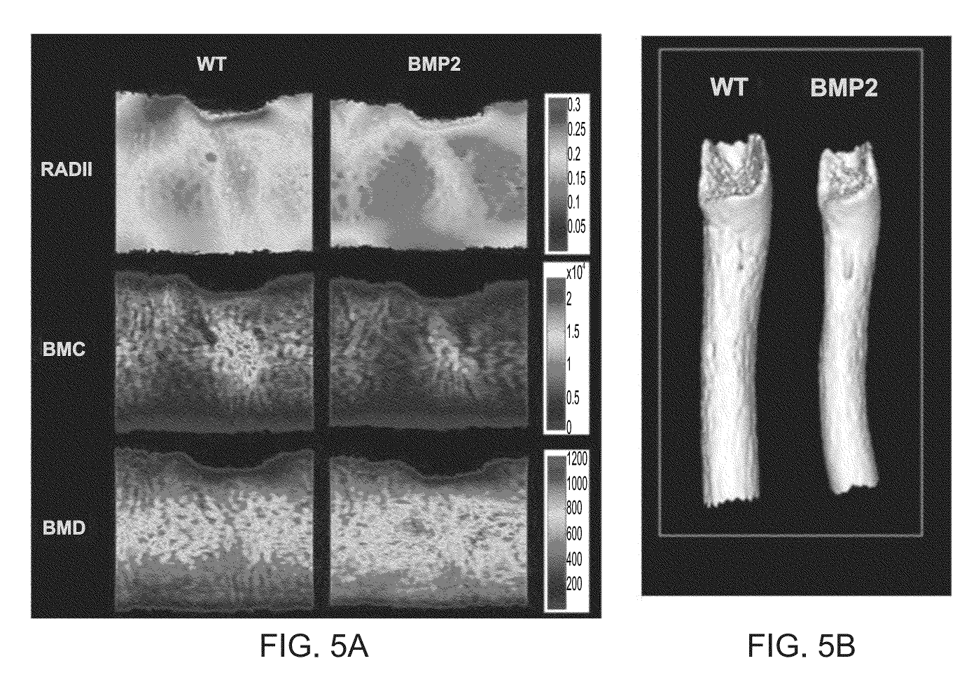

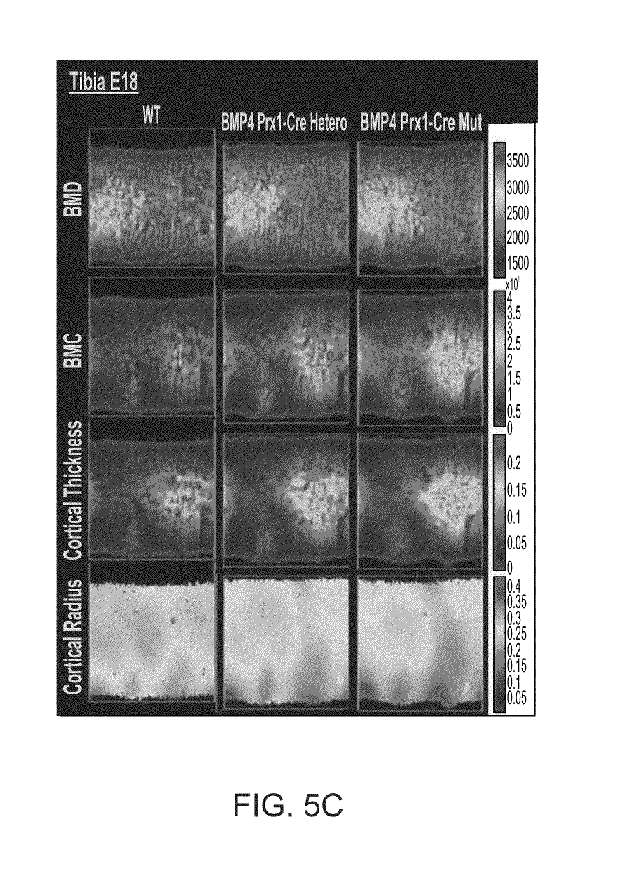

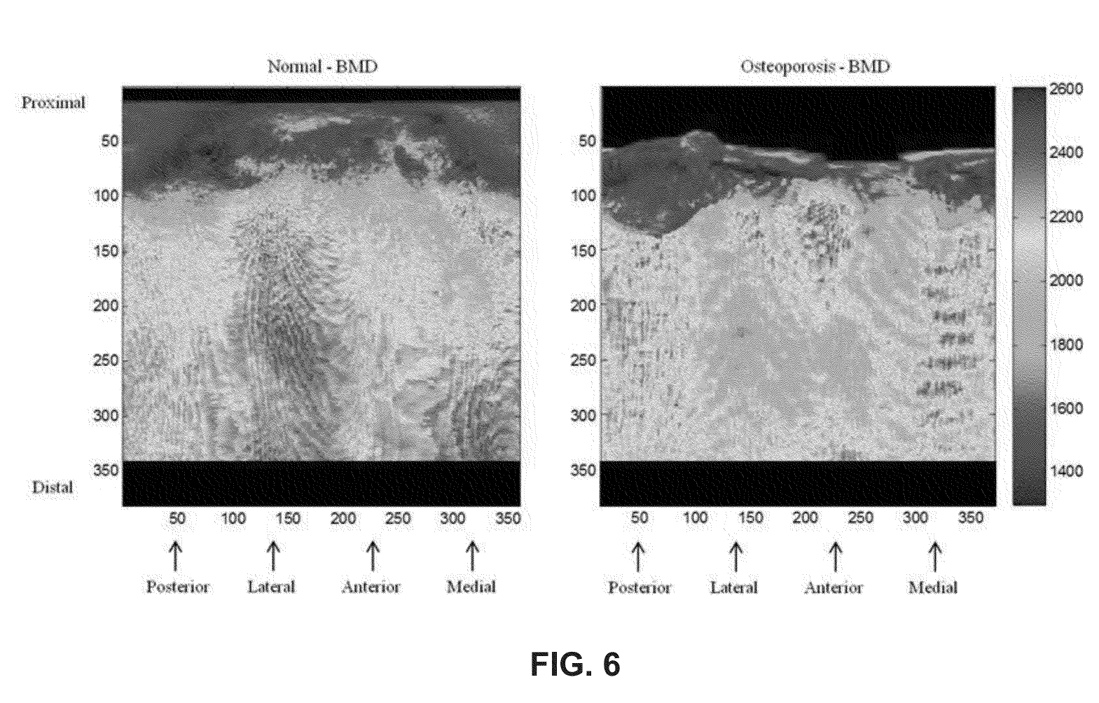

Reference is now made to FIG. 5A and FIG. 5C, which depict exemplary coded images, also referred to as unrolled image maps, which are generated using different operators, for example as described above, according to some embodiments of the present invention. FIG. 5A depicts unrolled image maps which are based on unrolled images of the Ulna bones of a wild type (WT) mouse and a bone morphogenetic protein 2 (BMP2) conditional knockout (K.O.) mouse at embryonic day 17, such as the bones depicted in FIG. 5B. It should be noted that BMPs are a group of growth factors which are known for their ability to induce ectopic bone formation. In addition, BMPs are also known to participate in the process of bone development. The molecular mechanisms by which BMPs act and their influence on the general morphology and mineralization of developing bones is still not fully understood.

In particular, the right side unrolled image maps are based on an unrolled image of an imaged bone of a transgenic mouse with inactivated BMP2 expressions in a limb-specific manner prior to the onset of skeletal development, referred to herein as BMP2 bone. The left side unrolled image maps are based on an unrolled image of an imaged bone of a WT mouse, referred to herein as WT bone. The unrolled image maps explicitly depict significant differences:
1. The Periosteal Thickness is significantly higher in WT bones, thus indicating a morphological effect of the mutation.
2. The BMC of the WT bone is significantly higher in most of its sectional regions than in respective sectional regions of the BMP2 bone.
3. The BMD of the WT bone is significantly lower in a significant portion of its sectional regions than in respective sectional regions of the BMP2 bone.
4. The spread of high density regions (BMD—in red) is relatively scattered in the WT bone, where in the BMP2 bone it tends to aggregate in two primary regions.

Collectively, the coded images depicted in FIG. 5A show the influence of the BMP2 gene on the morphology, the mineral density, and the spatial organization of mineral in embryonic bones. This indicates that BMP2 has a role in skeletal patterning. It should be noted that in 2006 a group of researchers from Harvard University has published a letter in Nature Genetics [19], describing the results of a study performed on BMP2, in which transgenic mice with inactivated BMP2 expression in a limb-specific manner prior to the onset of skeletal development, have been tested for their potential to initiate an endogenous bone repair response after having been fractured. In the letter, they state the following: “Bmp²^{^{c/c}}; Prx1::cre mice have few skeletal abnormalities at birth, allowing us to conclude that BMP2 expression during embryogenesis is not required for events that determine proper skeletal patterning, early limb chondrogenesis or the appropriate transition from cartilage to bone that occurs before birth”, and: “As newborn mice lacking limb expression of Bmp2 had normal skeletons, it is likely that other BMPs present in the developing limb can compensate for the loss of BMP2”. These statements emphasize the ability of our method to clearly identify and visualize osteo-characteristics that are otherwise undetectable or can hardly be noticed.

BMP4 is a member of the previously described BMP group. A transgenic mouse with inactivated expression of the BMP4 gene in a limb-specific manner prior to the onset of skeletal development will result in the absence of important skeletal elements like the Deltoid Tuberosity of the Humerus bone and the Lesser Trochanter of the Femur bone, yet no influence of BMP4 on the general morphology or mineralization of the developing bone has been reported so far. The coded images depicted in FIG. 5C show the influence of a complete conditional K.O. and of a removal of a single copy of the Bone Morphogenetic Protein (BMP4) gene on the morphology, the mineral density, and the spatial organization of mineral in Tibia bones of mice at the age of embryonic day 18 (E18) which have the following genetic conditions (from the first to the third columns on the left): (a) WT, (b) BMP4 Prx1-Cre conditional Heterozygous, and (c) BMP4 Prx1-Cre conditional K.O. The following can be view from the coded images:
1. The BMD is somehow increased in conditional K.O. mouse, and while in WT mouse there are quite a few “mineral holes” in which no mineral exists at all (color-coded black points scattered mostly on the left side of the map), almost no such holes are evident in the map of the conditional K.O. mouse.
2. BMC is significantly increased in the conditional K.O. mouse, especially around the right mid section of the map.
3. Cortical Thickness (the estimated distance between periosteum and the endosteum of the bone) is significantly increased in conditional K.O. mouse, especially around the right mid section of the map.
4. The Cortical Radius of the bone (distance from the center of the marrow to the periosteum) is significantly increased in the conditional K.O. mouse, meaning that the conditional K.O. bone is much wider than the WT bone.
5. As can be seen in all the images, by the distance between the upper ends of the maps and the lower ends (the fraction of Y axis segment that is occupied by the maps), the conditional K.O. bone is longer than the WT bone.

In all cases, the conditional heterozygous bone presents phenotypes that from a visual inspection, suggest higher similarity to the conditional K.O. mouse than to the WT mouse.

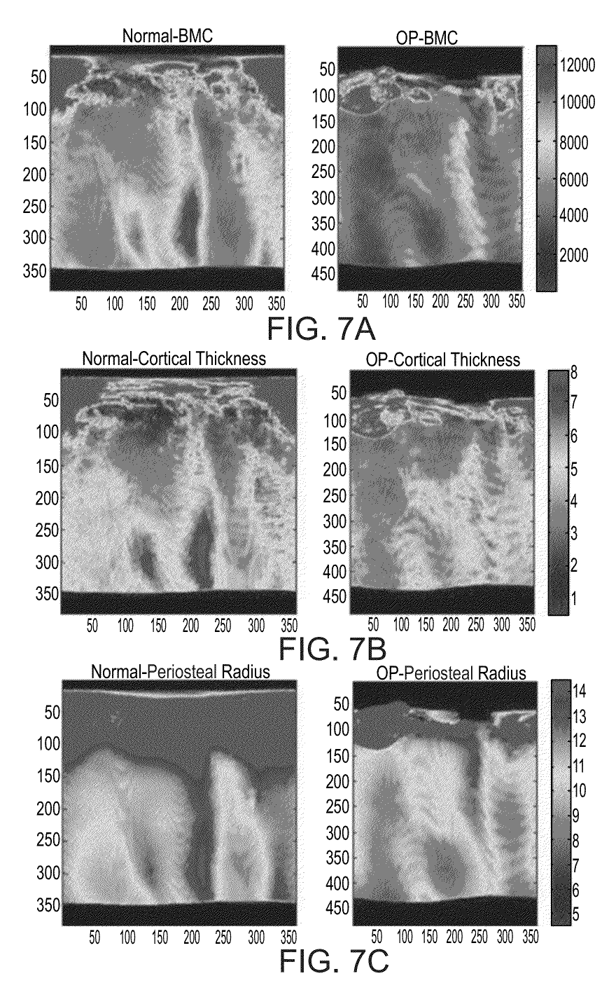

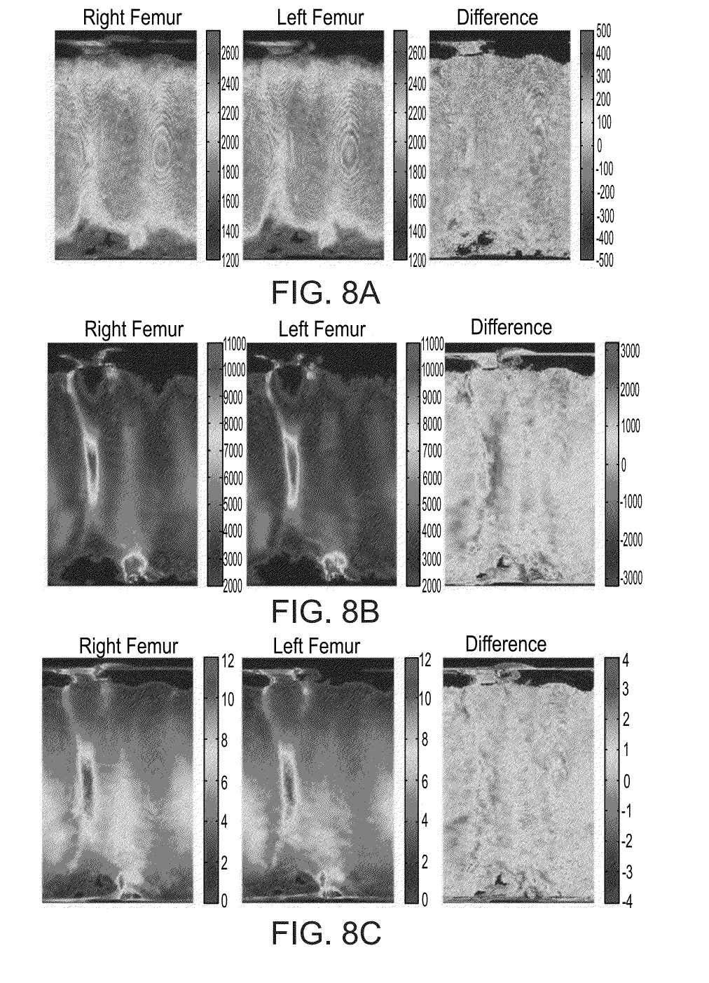

According to some embodiments of the present invention, the osteoporotic level of a Human bone may be diagnosed according to one or more of the generated coded images. For example, reference is now made to FIG. 6, which is a set of two exemplary unrolled image maps which are indicative of BMD, generated as described above, according to some embodiments of the present invention. The left and right unrolled image maps are based on an unrolled image generated from an image of a bone segment that is located at the first 3.5 cm of the proximal side of a Human Humerus bone. The left unrolled image map is based on an unrolled image of a bone segment of a 21 years old male (left) and the right unrolled image map is based on an unrolled image of a bone segment of an osteoporotic (OP) 83 years female. FIG. 6 depicts a color-bar which indicates the correlation between the radio-density in Hounsfield scale and the used colors. The normal Humerus presents significantly higher overall levels of BMD than the OP Humerus. In addition, a closer look at the localized level between the normal and the OP bone reveals a negative correlation between the densities. Regions of high mineral density in the normal bone express low mineral density in the OP bone and vice-verse, for instance around the lateral direction and between 10 mm and 30 mm (between Y=100 and Y=300), suggesting that Osteoporosis, in addition to causing a significant decrease in the general BMD also induce fundamental alterations in the pattern/spread of BMD. FIGS. 7A, 7B, and 7C depict exemplary unrolled image maps which are generated using different operators, according to some embodiments of the present invention. FIG. 7A depicts the BMC, in HU scale, in normal and OP bones FIG. 7B depicts the, Cortical thickness, in millimeters in normal and OP bones and 8C depicts the Periosteal Thickness, in millimeters, in normal and OP bones. As shown by these images, the spatial pattern of both morphological and mineral density parameters is significantly altered between the normal and the OP bones.

According to some embodiments of the present invention, the unrolled image maps may be used for diagnosing and studying osteo related pathologies in the bone segment.

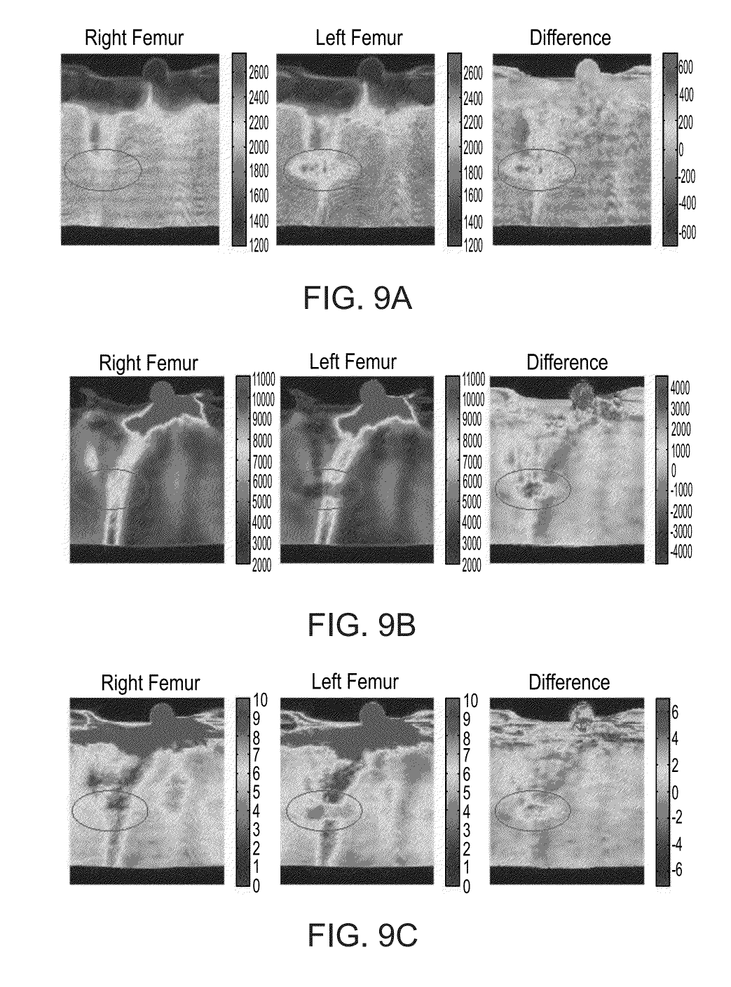

The diagnosis may be performed manually, for example by a user which reviews a print or a display of the unrolled image maps and/or automatically by a computing unit which process the unrolled image maps. Optionally, the diagnosis is based on a comparison between respective organs, for example between left and right bones and/or different segments of a diagnosed bone. Optionally, the diagnosis is based on a comparison between the unrolled image map of the diagnosed bone and exemplary unrolled image maps which are generated from one or more exemplary bones, healthy and/or pathological. The exemplary unrolled image maps may be based on a statistical analysis of a plurality of unrolled image maps of exemplary bones, optionally of patients having common characteristics, such as age, race, gender, weight, pathology, medical history and the like. Optionally, the unrolled image maps are diagnosed for detecting the presence and/or the absence of one or more metastatic sites or primary tumors. For example, reference is made to FIGS. 8A-8C and 9A-9C are unrolled image maps of left and right femur bones of a healthy patient and a cancerous patient, generated according to some embodiments of the present invention. Each one of these figures also includes a map that indicates the differences between the mapped osteo related characteristics in respective radial axes of the left and right femur bones. FIGS. 8A and 9A depict unrolled image maps indicative of BMD in the femur bones. FIGS. 8B and 9B depict unrolled image maps indicative of BMC in the femur bones. FIGS. 8C and 9C depict unrolled image maps indicative of cortical thickness in the femur bones. As shown at FIGS. 8A-8C the difference between the respective osteo related characteristics in the left and right femur bones is relatively minor in all their radial axes. However, in FIGS. 9A-9C the difference between the respective osteo related characteristics at the metastasis area is relatively high. In addition, the differences between the BMD values of radial axes of the left femur bone and the right femur bone are relatively high. The BMD values of the right femur bone, which is cancerous, are higher. Namely, contrary to the significant decrease in BMD at the metastatic site, the rest of the infected bone has a much higher BMD level at non-metastatic regions than the normal bone. This difference may be a normal reaction of the body to the presence of the metastasis and/or an outcome of a change in the posture and/or walking pattern of the patient.

As shown at 106, the process depicted in 102-105 may be repeated a plurality of time for all the cylindrical bones which are depicted in the received reconstructed volume image. In such a manner, all the long bones which are depicted in a 3D image, such as a CT image, for instance in a digital imaging and communications in medicine (DICOM) object, are analyzed.

The process depicted in FIG. 1 and the aforementioned automatic diagnosis may be performed automatically on reconstructed volume images, such as CT images, providing the physician and/or the radiologist and additional indication about the pathological state of the imaged bones. In such embodiments, the process may be implemented by a computer aided diagnosis (CAD) module that process reconstructed volume images, such as CT images, for example on a client terminal of a physician and/or a radiologist and/or on images stored in a central database, such as a DICOM server, a radiology information system (RIS), and a hospital information system (HIS), also known as a clinical information system (CIS).

Optionally, the unrolled image maps may be used to quantify and/or image the properties of one or more metastatic sites or primary tumors which identified therein and serve for staging the patient, for follow-ups during treatments, and for the identification of otherwise hardly visible instances of the disease. This platform may serve any bone related clinical situation that is evident in mineralization or morphology, other examples: skeletal traumas, Osteomalachia, Osteogenesis Imperfecta, and the like.

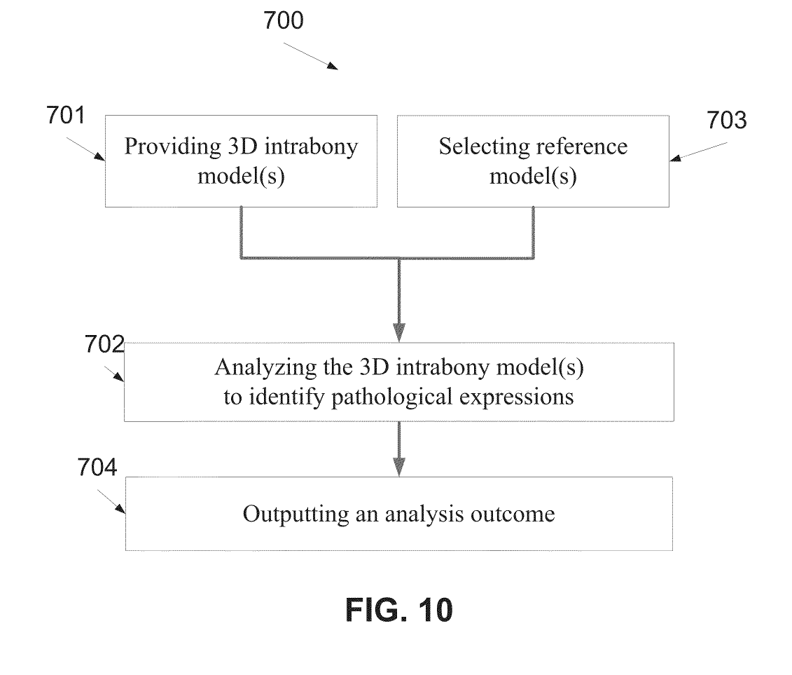

Reference is now made to FIG. 10, which is a flowchart 700 of a method of diagnosing an intrabony space, according to some embodiments of the present invention.

First, as show, at 701, one or more coded images each of one or more intrabody osteo related characteristics of one or more bones of patient, for example a coded image as depicted in FIG. 5A, are provided. Each coded image is optionally generated as described above with reference to FIG. 1. The osteo related characteristic may be a BMC, a BMD, the Cortical Thickness of the bone, and/or any other characteristics of the bone which may be extracted from the coded image, for example as described above. The coded image is optionally extracted from a segment depicting a bone of a patient in a reconstructed volume image, such as a CT image, for example as described above.

Now, as shown at 702, the one or more coded images are analyzed to identify in each one or more pathological expressions in patterns depicted in the coded image(s) and/or one or more local pathological indicators. The analysis is optionally performed, in each coded image, by matching between the pattern depicted in the coded image and a pattern depicted in a 3D reference map. The 3D reference map is optionally selected from a database which includes a plurality of reference models, as shown at 703. Each reference model depicts an exemplary pattern associated with a different demographic group of patients. The demographic groups may be divided according to different age, gender, race, pathology, and/or medical condition. The analysis of the coded image of the patient allows identifying local indicators in the different volumes of the depicted pattern. For example, Each one of FIGS. 9A-9C depicts a local indicator (encircled area) that is indicative of cancer. Such local indicators, which are optionally pathological expression of the one or more osteo related characteristics in different volumes of the depicted pattern, are very unlikely to be identified by summing data from the bone as a whole. The summing of data from the bone as a whole, for example by averaging, does not reflect pathological expressions in different volumes of the bone and does not allow detecting a pathology which has a local expression which is not reflected most of the intrabony space. Additionally or alternatively, the pattern depicted in the coded image may be analyzed to identify pathologies which have different expressions in different intrabony volumes. For example, as depicted in FIG. 5A, BMP2 may be identified by high BMD spots in specific areas of the bone. Such expressions, which are optionally pathological expressions of the one or more osteo related characteristics in the depicted pattern, are either unlikely or impossible to be identified by summing data from the bone as a whole. The summing of data from the bone as a whole, for example by averaging, ignores the pattern and as it relates only a sum of values and does not allow detecting a pathology which has a pattern not reflected by values of voxels from an image depicting the intrabony space.

According to some embodiments of the present invention, two coded images are separately generated for respective segments in respective left and right bones, for example as shown at FIGS. 8A-8C and 9A-9C. In such embodiments, patterns from the two coded images may be compared to allow identifying identity, similarity, and/or variation therebetween. For example, FIGS. 9A-9C depicts a repetition of the same local indicator in each of the two coded images.

Now, as shown at 704, the outcome of the analysis is outputted, for example displayed to a clinician on a display connected to a local client terminal, a thin terminal, and/or any other computing device. The outcome may also be documented in a medical database, such as PACS, RIS, CIS and/or HIS, optionally as another record of the patient, for example in association with a reconstructed volume from which the coded image is extracted.

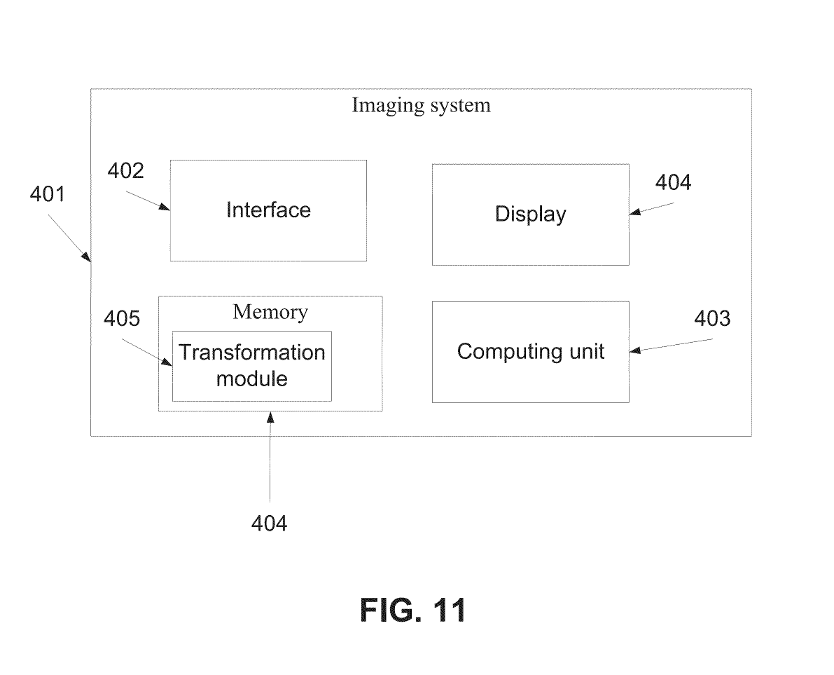

Reference is now made to FIG. 11, which is a schematic illustration of an imaging system 401 of imaging intrabony space of a cylindrical bone, for example by implementing the method depicted in FIG. 1, according to some embodiments of the present invention. The system 100 is optionally implemented on a network node, such as one or more servers and/or on a client terminal, such as a laptop, a tablet, a personal computer and the like. Optionally, the system 401 includes a computing unit 403 which executes one or more software applications and/or modules, referred to herein as a transformation module 405 for calculating the visualization of the intrabony space, as described above, and to generate one or more maps of osteo related characteristics accordingly. Optionally, the computing unit 403 comprises and/or connected to a memory 404 which is set to store the respective software applications and/or modules. The system includes an interface 402 which receives a reconstructed volume image depicting a cylindrical bone, for example as described bone. The interface is optionally a network interface for receiving the image via to a computer network, such as a local area network (LAN) and/or a wide area network (WAN). The network interface 402 optionally comprises a wireless network interface controller (WNIC) which connects to a radio-based computer network, optionally based on IEEE 802.11 standards. Additionally or alternatively, the network interface 402 optionally comprises a network interface controller (NIC) which connects to a wire-based network such as token ring or Ethernet. In such embodiments, the computing unit 403 transforms a segment depicting the cylindrical bone in the reconstructed volume image to generate a 3D model which aligns a representation of each of a plurality of radial axes having a plurality of different angular orientations of the cylindrical bone in parallel to a common axis, for example by performing a transformation as described above and depicted in FIGS. 2A and 2B. The computing unit 403 generates maps which map osteo related characteristic(s) of the cylindrical bone along various radial axes as described above. The system 401 further includes a display 404 which presents the maps, for example an LCD monitor and/or a projector. Optionally, the interface 402 receives reconstructed volume image from a central database as described above.

It is expected that during the life of a patent maturing from this application many relevant systems and methods will be developed and the scope of the term a computing unit, a reconstructed volume image, and a display is intended to include all such new technologies a priori.

As used herein the term “diagnose” may refer to the act of “bone analysis”.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of imaging and/or diagnosing intrabony space, comprising:
providing a reconstructed volume image depicting a cylindrical bone;
identifying a segment depicting said cylindrical bone in said reconstructed volume image;
transforming said segment to generate a three-dimensional (3D) model which aligns a representation of each of a plurality of radial portions having a plurality of different angular orientations of said cylindrical bone in parallel to a common axis;
performing a calculation of at least one osteo related characteristic of said cylindrical bone along each said radial portion using said 3D model, said at least one at least one osteo related characteristic comprises at least one of unweighted bone mineral content (uwBMC) of said cylindrical bone along each said radial portion and bone mineral density (BMD) of said cylindrical bone along each said radial portion;
generating, based on said calculation, at least one coded image mapping of said at least one osteo related characteristic of said cylindrical bone along each said radial portion; and
outputting said at least one coded image.

2. The method of claim 1, wherein said at least one coded image is color coded.

3. (canceled)

4. The method of claim 1, further comprising analyzing said at least one coded image to automatically estimate at least one osteo related pathology.

5. The method of claim 1, further comprising registering at least said segment onto a nonlinear cylindrical space, said transforming comprising mapping said plurality of radial portions by a transformation from an continuous nonlinear cylindrical space to a continuous Euclidean space.

6-10. (canceled)

11. The method of claim 1, wherein said generating comprising calculating the thickness of the Endosteum along each said radial portion.

12. (canceled)

13. The method of claim 1, wherein said generating comprising calculating the distance from the center of the Bone marrow to at least one of the Periosteum along each said radial portion and the Endosteum along each said radial portion.

14. The method of claim 1, wherein said generating comprising calculating at least one of cortical porosity and unweighted cortical porosity along each said radial portion.

15. (canceled)

16. The method of claim 1, wherein said generating comprising calculating said at least one osteo related characteristic along each said radial portion as a function of a plurality of radiodensity units of respective voxels in at least said segment.

17. (canceled)

18. The method of claim 1, wherein said at least one osteo related characteristic comprises morpho-density characteristics.

19. The method of claim 1, further comprising diagnosing an osteoporotic level of said cylindrical bone by analyzing said at least one coded image.

20. The method of claim 1, further comprising detecting at least one metastatic or primary tumor site in said cylindrical bone by analyzing said map.

21. The method of claim 1, wherein said plurality of radial portions are perpendicular to a longitudinal axis of said cylindrical bone.

22. The method of claim 1, wherein said transforming emulates the spreading of the cortex layer of said segment on a plane.

23. The method of claim 1, wherein said reconstructed volume image depicting a plurality of cylindrical bones said identifying, transforming, generating, and outputting are iteratively repeated with each said cylindrical bone.

24. A system of imaging intrabony space, comprising:
an interface which receives a reconstructed volume image depicting a cylindrical bone;
a computing unit which transforms a segment depicting said cylindrical bone in said reconstructed volume image to generate a three-dimensional (3D) model which aligns a representation of each of a plurality of radial axes having a plurality of different angular orientations of said cylindrical bone in parallel to a common axis, performs a calculation of at least one osteo related characteristic of said cylindrical bone along each said radial portion using said 3D model, said at least one osteo related characteristic comprises at least one of unweighted bone mineral content (uwBMC) of said cylindrical bone along each said radial portion and bone mineral density (BMD) of said cylindrical bone along each said radial portion, and generates at least one coded image mapping of said at least one osteo related characteristic of said cylindrical bone along each said radial portion; and
a display which outputs said at least one coded image.

25. The system of claim 20, wherein said interface receives said reconstructed volume image from a member of a group consisting of: a computer aided diagnosis (CAD) module, a client terminal of a physician, a central database, a DICOM server, a radiology information system (RIS), a hospital information system (HIS), and a clinical information system (CIS).

26. A method of analyzing an intrabony space, comprising:
providing at least one coded image mapping a sum of values of at least one osteo related characteristic in each of a plurality of portions of at least one bone of a patient;
analyzing a pattern depicted in said at least one coded image to identify a pathological expression; and
outputting an outcome of said analysis.

27. (canceled)

28. The method of claim 22, wherein said plurality of portions having a plurality of different angular orientations in parallel to a common axis.

29. (canceled)

30. The method of claim 22, wherein said analyzing comprises:
selecting at least one 2D reference map according to at least one demographic characteristic of said patient, and
matching between said at least one 2D reference map and said at least one coded image.

31. The method of claim 22, wherein said at least one coded image comprises a plurality of coded images, said analyzing comprises matching between said plurality of coded images to identify a difference and performing said analysis according to said difference.
